(19) United States
(12) Plant Patent Application Publication
(10) Pub. No.: US 2015/0373892 P1
(43) Pub. Date: Dec. 24, 2015

(54) Calibrachoa plant named 'PAS1020342'

(50) ;

(71) Applicant: Ball Horticultural Company, West Chicago, IL (US)

(72) Inventor: Jason Jandrew, Nipomo, CA (US)

(21) Appl. No.: 14/120,751

(22) Filed: Jun. 23, 2014

(51) Int. Cl. A01H 5/00

(52) U.S. Cl. PLT/413

(57) ABSTRACT

A new and distinct cultivar of Calibrachoa plant named ‘PAS1020342’, characterized by its dark red-colored flowers, medium green-colored foliage, and moderately vigorous, mounded-trailing growth habit, is disclosed.

### LATIN NAME OF GENUS AND SPECIES OF PLANT CLAIMED

Calibrachoa×hybrida

### VARIETY DENOMINATION

‘PAS1020342’

### BACKGROUND OF THE INVENTION

The present invention relates to a new and distinct cultivar of Calibrachoa plant botanically known as Calibrachoa×hybrida and hereinafter referred to by the cultivar name ‘PAS1020342’.

The new cultivar originated in a controlled breeding program in Elburn, Ill. during May 2011. The objective of the breeding program was the development of Calibrachoa cultivars with unique flower coloration and mounded-trailing growth habit.

The new Calibrachoa cultivar is the result of cross-pollination. The female (seed) parent of the new cultivar is the proprietary Calibrachoa×hybrida breeding selection coded N3654R, not patented, characterized by its medium red-colored flowers, medium green-colored foliage, and moderately vigorous, spreading growth habit. The male (pollen) parent of the new cultivar is the proprietary Calibrachoa×hybrida breeding selection coded N8924R, not patented, characterized by its medium yellow-colored flowers, medium green-colored foliage, and moderately vigorous, semi-upright growth habit. The new cultivar was discovered and selected as a single flowering plant within the progeny of the above stated cross-pollination during August 2011 in a controlled environment in Elburn, Ill.

Both parents have a degree of homozygosity such that the progeny of the cross N3654R times N8924R are phenotypically uniform and essentially identical to ‘PAS1020342’. Accordingly, phenotypically identical ‘PAS1020342’ can be produced by sexual reproduction as well as asexual reproduction.

Asexual reproduction of the new cultivar by terminal stem cuttings since August 2011 in West Chicago, Ill. has demonstrated that the new cultivar reproduces true to type with all of the characteristics, as herein described, firmly fixed and retained through successive generations of such asexual propagation.

### SUMMARY OF THE INVENTION

The following characteristics of the new cultivar have been repeatedly observed and can be used to distinguish ‘PAS 1020342’ as a new and distinct cultivar of Calibrachoa plant:

1. Dark red-colored flowers;

2. Medium green-colored foliage;

3. Moderately vigorous, mounded-trailing growth habit.

Plants of the new cultivar differ from plants of the female parent primarily in growth habit. Plants of the new cultivar differ from plants of the male parent primarily in flower color and growth habit.

Of the many commercially available Calibrachoa cultivars, the most similar in comparison to the new cultivar is CABARET Bright Red ‘Balcabrite’, U.S. Plant Pat. No. 24,401. However, in side by side comparisons, plants of the new cultivar differ from plants of ‘Balcabrite’ in at least the following characteristics:

1. Plants of the new cultivar have fewer flowers per plant than plants of ‘Balcabrite’; and

2. Plants of the new cultivar have fewer main branches than plants of ‘Balcabrite’.

### BRIEF DESCRIPTION OF THE PHOTOGRAPHS

The accompanying photographs show, as nearly true as it is reasonably possible to make the same in color illustrations of this type, typical flower and foliage characteristics of the new cultivar. Colors in the photographs differ slightly from the color values cited in the detailed description, which accurately describes the colors of ‘PAS1020342’. The plants were grown in 4.5-inch pots for 5 weeks in a greenhouse in West Chicago, Ill. Plants were given one pinch one week before transplant.

FIG. 1 illustrates a side view of the overall growth and flowering habit of ‘PAS 1020342’.

FIG. 2 illustrates a close-up view of an individual flower of ‘PAS1020342’.

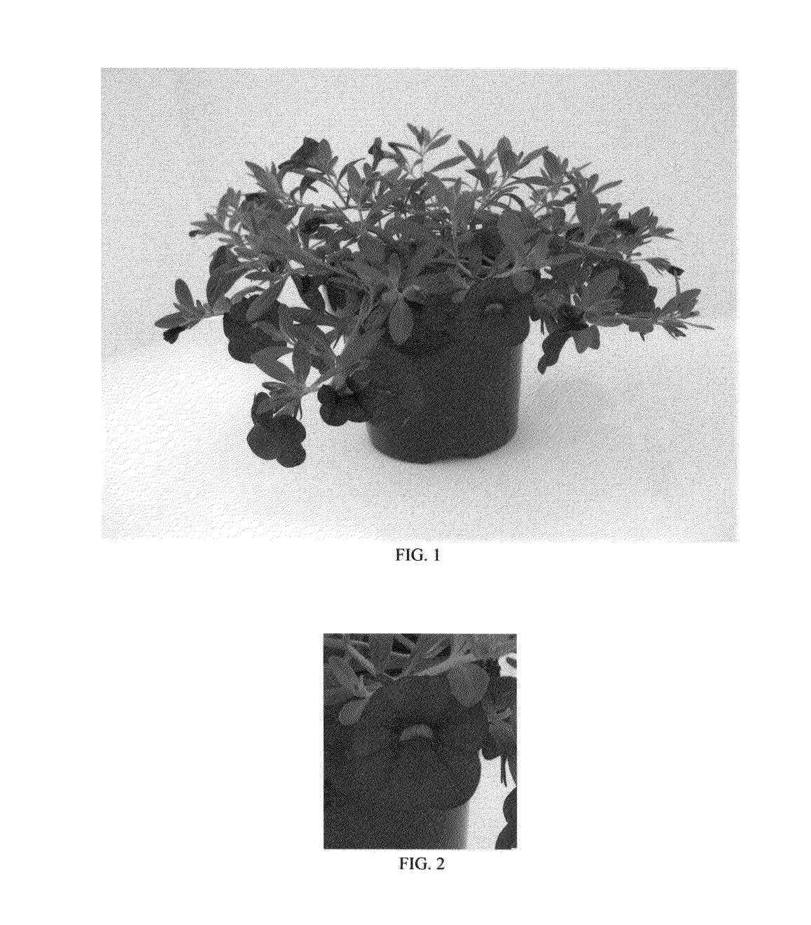

### DETAILED BOTANICAL DESCRIPTION

The new cultivar has not been observed under all possible environmental conditions to date. Accordingly, it is possible that the phenotype may vary somewhat with variations in the environment, such as temperature, light intensity, and day length, without, however, any variance in genotype.

The chart used in the identification of colors described herein is The R.H.S. Colour Chart of The Royal Horticultural Society, London, England, 2007 edition, except where general color terms of ordinary significance are used. The color values were determined in June 2014 under natural light conditions in West Chicago, Ill.

The following descriptions and measurements describe plants produced from cuttings from stock plants and grown in a glass-covered greenhouse under conditions comparable to those used in commercial practice. The plants were grown in West Chicago, Ill. in 4.5-inch pots for 5 weeks utilizing a soilless growth medium. Plants were given one pinch one week before transplant. Greenhouse temperatures were maintained at approximately 66° F. to 70° F. (19° C. to 21° C.) during the day and approximately 58° F. to 62° F. (14° C. to 17° C.) during the night. Greenhouse light levels of 2,500 footcandles to 6,000 footcandles were maintained during the day. Measurements and numerical values represent averages of typical plants.
Botanical classification: Calibrachoa×hybrida cultivar PAS1020342.
Parentage:
   Female parent.—Proprietary Calibrachoa×hybrida breeding selection coded N3654R, not patented.
   Male parent.—Proprietary Calibrachoa×hybrida breeding selection coded N8924R, not patented.
Propagation:
   Type cutting.—Terminal stem.
   Time to initiate roots.—Approximately 6 to 8 days.
   Time to produce a rooted cutting.—Approximately 24 to 28 days.
   Root description.—Fibrous.
   Rooting habit.—Freely branching.
Plant description:
   Commercial crop time.—Approximately 4 to 6 weeks from a rooted cutting to finish in a 10 cm pot.
   Growth habit and general appearance.—Moderately vigorous, mounded-trailing.
   Size.—Height from soil level to top of plant plane: Approximately 8.0 cm. Width: Approximately 29.3 cm.
   Branching habit.—Freely branching, pinching enhances basal branching. Quantity of main branches per plant: Approximately 3.
   Branch.—Strength: Strong, flexible. Length: Approximately 16.9 cm. Diameter: Approximately 2.0 mm. Length of central internode: Approximately 1.7 cm. Texture: Densely glandular pubescent. Gland color: Colorless, transparent. Color of young and mature stems: 144A.
Foliage description:
   General description.—Quantity of leaves per main branch: Approximately 12. Fragrance: Slight. Form: Simple. Arrangement on flowering stem: Opposite.
   Leaves.—Aspect: At acute angle to stem with tip turning downward. Shape: Elliptic. Margin: Entire. Apex: Broadly acute. Base: Attenuate. Venation pattern: Pinnate. Length of mature leaf: Approximately 3.7 cm. Width of mature leaf: Approximately 1.3 cm. Texture of upper and lower surfaces: Moderately glandular pubescent. Gland color: Colorless, transparent. Color of upper surface of young and mature foliage: 137A with midvein of 144D. Color of lower surface of young and mature foliage: Closest to 138B with midvein of 138C.
   Petiole.—Length: Approximately 6.0 mm. Diameter: Approximately 2.0 mm. Texture: Moderately glandular pubescent. Gland color: Colorless, transparent. Color: 144D.
Flowering description:
   Flowering habit.—‘PAS 1020342’ is freely flowering under outdoor growing conditions with substantially continuous blooming from spring through autumn and year-round in greenhouse environment.
   Lastingness of individual flower on the plant.—Approximately 5 to 7 days.
Flower description:
   General description.—Type: Single, salverform. Quantity per plant: Approximately 17. Fragrance: None detected.
   Bud.—Rate of opening: Generally takes 3 to 4 days for bud to progress from first color to fully open flower. Quantity per plant: Approximately 10.
   Bud just before opening.—Shape: Oblong. Length: Approximately 1.7 cm. Diameter: Approximately 5.0 mm. Texture: Densely glandular pubescent. Gland color: Colorless, transparent. Color: Petal portion N79B and tube portion 151C, venation of N186A.
   Corolla.—Diameter: Approximately 3.2 cm.
   Petals.—Quantity: 5, fused to form a tube. Shape: Obovate. Margin: Entire. Apex: Obtuse. Length from tube: Approximately 1.4 cm. Length of free portion: Approximately 5.0 mm. Width: Approximately 1.5 cm. Texture of upper surface: Glabrous. Texture of lower surface: Glandular pubescent, dense along venation. Gland color: Colorless, transparent. Color of upper surface when first open: Closest to 53A with midveins of N186A. Color of lower surface when first and fully open: 185C with midveins of N186A. Color of upper surface when fully open: Closest to 53A to 53B with midveins of N186A.
   Corolla tube.—Length: Approximately 1.7 cm. Diameter at distal end: Approximately 6.0 mm. Diameter at proximal end: Approximately 1.0 mm. Texture of inner surface: Glabrous. Texture of outer surface: Densely glandular pubescent. Gland color: Colorless, transparent. Color of inner surface: 13A with venation of N186A. Color of outer surface: 151C with venation of N186A.
   Sepals.—Quantity per flower: 5, fused along lower half. Shape: Lanceolate. Apex: Acute. Length: Approximately 1.5 cm. Width: Approximately 3.0 mm. Texture of upper and lower surfaces: Densely glandular pubescent. Gland color: Colorless, transparent. Color of upper surface: 137A. Color of lower surface: 137B with base of 144B.
   Peduncle.—Strength: Strong, flexible. Aspect: Acute angle to stem. Length: Approximately 1.8 cm. Diameter: Approximately 1.0 mm. Texture: Densely glandular pubescent. Gland color: Colorless, transparent. Color: 144A.
   Reproductive organs.—Androecium: Stamen quantity: 5, basifixed. Stamen length: Approximately 1.1 cm. Filament length of fixed portion: Approximately 6.0 mm. Filament color: 154D. Anther shape: Bilobed, ovoid. Anther length: Approximately 1.0 mm. Anther color: 8A. Pollen amount: Abundant. Pollen color: 8C. Gynoecium: Pistil quantity: 1 per flower. Pistil length: Approximately 1.2 cm. Stigma shape: Funnel. Stigma length: Less than 1.0 mm. Stigma color: 144A. Style length: Approximately 1.0 cm. Style color: N144D. Ovary length: Approximately 2.0 mm. Ovary color: N144C.
Seed and fruit production: Neither seed nor fruit production has been observed.
Disease and pest resistance: Resistance to pathogens and pests common to Calibrachoa has not been observed.

What is claimed is:

1. A new and distinct cultivar of Calibrachoa plant named ‘PAS1020342’, substantially as herein shown and described.
